(19) United States
(12) Patent Application Publication
(10) Pub. No.: US 2021/0212580 A1
(43) Pub. Date: Jul. 15, 2021

(54) BLOOD PRESSURE MEASUREMENT DEVICE

(71) Applicants: OMRON HEALTHCARE Co., Ltd., Kyoto (JP); OMRON Corporation, Kyoto (JP)

(72) Inventors: Tomoyuki NISHIDA, Kyoto (JP); Takashi ONO, Kyoto (JP); Yuichiro ARIMA, Kyoto (JP); Takayuki MATSUOKA, Kyoto (JP)

(21) Appl. No.: 17/216,878

(22) Filed: Mar. 30, 2021

Related U.S. Application Data
(63) Continuation of application No. PCT/JP2019/038367, filed on Sep. 27, 2019.

(30) Foreign Application Priority Data
Oct. 15, 2018 (JP) 2018-194347

(51) Int. Cl. A61B 5/022

(52) U.S. Cl. CPC A61B 5/02233; A61B 5/681

(57) ABSTRACT

A blood pressure measurement device includes a cuff structure formed of a resin material and configured to be inflated with a fluid, and a curler curved in such a manner as to follow along a circumferential direction of a portion of a living body where the blood pressure measurement device is attached, the curler being formed with a first end and a second end spaced apart from each other, the cuff structure being welded to the curler, and a portion of the curler where the cuff structure is welded being formed of a material similar to the resin material forming the cuff structure.

### CROSS-REFERENCE TO RELATED APPLICATIONS

This application is the U.S. national stage application filed pursuant to 35 U.S.C. 365(c) and 120 as a continuation of International Patent Application No. PCT/JP2019/038367, filed Sep. 27, 2019, which application claims priority from Japanese Patent Application No. 2018-194347, filed Oct. 15, 2018, which applications are incorporated herein by reference in their entireties.

### TECHNICAL FIELD

The present invention relates to a blood pressure measurement device for measuring blood pressure.

### BACKGROUND ART

In recent years, blood pressure measurement devices for measuring blood pressure are being used to monitor health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

As such a blood pressure measurement device, for example, a so-called integral type is known in which a cuff is integrated with a device body feeding a fluid to the cuff. Such blood pressure measurement devices pose a problem in that wrinkles, folds, or the like in the cuff reduce the accuracy of measurement results for the measured blood pressure. Additionally, in the blood pressure measurement device, the cuff needs to be inflated in the direction in which the blood vessels are occluded and to closely contact the wrist.

Thus, a technique for a blood pressure measurement device is known in which a curler is used between a belt and the cuff to bring the cuff inflated into close contact with the upper arm or the wrist as disclosed in JP 2018-102743 A. The curler has a shape along the circumferential shape of the upper arm or the wrist, for example, and the cuff is disposed on the inner circumferential surface of the curler. Furthermore, the curler is constituted by using a relatively hard resin material that is deformed in such a manner as to conform to the circumferential shape and thickness of the upper arm or the wrist due to tightening of the belt when the blood pressure measurement device is attached and that can be inhibited from being deformed in spite of inflation of the cuff.

Such curlers allow the cuff to suitably compress the wrist when the cuff is inflated and concentrate the bulge of the cuff in the direction in which the blood vessels are occluded. Additionally, the curler prevents wrinkles, folds, and the like from occurring in the cuff.

In addition, known methods for joining the curler and the cuff include junction using a bonding layer such as a double-sided tape or an adhesive and junction such as sewing and riveting which uses another member.

### CITATION LIST

### Patent Literature

Patent Document 1: JP 2018-102743 A

### SUMMARY OF INVENTION

### Technical Problem

For the above-described blood pressure measurement device, wearable devices attached to the wrist have recently been proposed. Such a blood pressure measurement device of a wearable device is required to be further miniaturized. In particular, assuming that a blood pressure monitor is always worn and used, the blood pressure measurement device is required to be as small as a wristwatch.

However, in a junction using a bonding layer or another member, a junction margin needs to be provided on the cuff or the curler, leading to an increase in the size and shape of the cuff or the curler. In this way, the size and shape of the blood pressure measurement device are increased due to the bonding layer or another member, making miniaturization of the blood pressure measurement device difficult.

Thus, an object of the present invention is to provide a blood pressure measurement device that can be miniaturized.

### Solution to Problem

According to one aspect, a blood pressure measurement device is provided that includes a cuff structure formed of a resin material and configured to be inflated with a fluid, and a curler curved in such a manner as to follow along a circumferential direction of a portion of a living body where the blood pressure measurement device is attached, the cuff structure being welded to the curler, and a portion of the curler where the cuff structure is welded being formed of a material similar to a resin material forming the cuff structure.

Here, the fluid includes a liquid and air. The cuff refers to a member that is wrapped around the upper arm, the wrist, or the like of a living body when the blood pressure is measured and that is inflated by being fed with the fluid. The cuff includes a bag-like structure such as an air bag.

Furthermore, “similar materials” refer to two materials that are highly compatible with each other in thermal welding and that have the same softening temperature or close softening temperatures. “Compatibility” refers to the degree of mixing of the resin materials softened or melted during welding, and “high compatibility” means that a junction can be achieved in which the resin materials softened or melted during welding mix together at a suitable degree, that is, a junction can be achieved at a required junction strength.

According to this aspect, the cuff structure and the curler can be suitably joined by thermal welding, thus allowing the cuff structure to be rigidly joined to the curler. In addition, since the cuff structure and the curler can be joined directly by thermal welding, a separate junction margin need not be provided, and the cuff structure and the curler need not be joined using another member as in sewing or the like. This allows prevention of an external shape from being enlarged due to the junction margin or another member, thus enabling the blood pressure measurement device to be miniaturized.

In the blood pressure measurement device according to the one aspect described above, the blood pressure measurement device is provided in which the curler is formed of a material similar to the resin material constituting the cuff structure.

According to this aspect, the curler can be formed of a single type of resin material, making manufacturing easier. Additionally, all portions of the curler can be thermally welded to the cuff structure, and thus the welding portion can be designed at a high degree of freedom.

In the blood pressure measurement device according to the one aspect described above, the blood pressure measurement device is provided in which the curler includes a first portion provided in a portion welded to the cuff structure, the first portion constituted by a material similar to the material constituting the cuff structure, and a second portion formed integrally with the first portion and constituted by a material harder than the material of the first portion.

According to this aspect, the curler can be thermally welded to the cuff structure by using the first portion, and a function required for the curler is obtained using a second portion, leading to a high degree of freedom for selection of the material.

In the blood pressure measurement device according to the one aspect described above, the blood pressure measurement device is provided in which the cuff structure is welded to an inner circumferential surface of the curler.

According to this aspect, even in a case where the cuff structure is equal to or smaller than the curler in a widthwise dimension, the cuff structure can be joined to the curler, allowing the blood pressure measurement device to be miniaturized.

In the blood pressure measurement device according to the one aspect described above, the blood pressure measurement device is provided in which the cuff structure includes, at a widthwise edge, a junction piece welded to a part of an outer circumferential surface of the curler, and is disposed on an inner circumferential surface of the curler.

According to this aspect, the junction piece can be provided on a portion of the cuff structure, and the junction piece can be folded back toward the outer circumferential surface of the curler and joined to the outer circumferential surface of the curler. Thus, even when the cuff structure is welded to the outer circumferential surface of the curler, an increase in the widthwise dimension of the curler can be suppressed, enabling the blood pressure measurement device to be miniaturized.

### Advantageous Effects of Invention

The present invention can provide a blood pressure measurement device that can be miniaturized.

### BRIEF DESCRIPTION OF DRAWINGS

FIG. 1 is a perspective view illustrating a configuration of a blood pressure measurement device according to a first embodiment of the present invention.

FIG. 2 is a perspective view illustrating the configuration of the blood pressure measurement device.

FIG. 3 is an exploded perspective view illustrating the configuration of the blood pressure measurement device.

FIG. 4 is an explanatory diagram illustrating a state in which the blood pressure measurement device is attached to the wrist.

FIG. 5 is a block diagram illustrating the configuration of the blood pressure measurement device.

FIG. 6 is a perspective view illustrating a configuration of a device body and a curler of the blood pressure measurement device.

FIG. 7 is a plan view illustrating a configuration of a cuff structure of the blood pressure measurement device.

FIG. 8 is a plan view illustrating another configuration of the cuff structure of the blood pressure measurement device.

FIG. 9 is a cross-sectional view illustrating a configuration of a belt, the curler, and the cuff structure of the blood pressure measurement device.

FIG. 10 is a cross-sectional view illustrating the configuration of the curler and the cuff structure of the blood pressure measurement device.

FIG. 11 is a cross-sectional view illustrating the configuration of the curler and the cuff structure of the blood pressure measurement device.

FIG. 12 is an explanatory diagram illustrating the configuration in which the cuff structure is inflated in a state in which the blood pressure measurement device is attached to the wrist.

FIG. 13 is a cross-sectional view illustrating the configuration in which the cuff structure is inflated in a state in which the blood pressure measurement device is attached to the wrist.

FIG. 14 is a flowchart illustrating an example of usage of the blood pressure measurement device.

FIG. 15 is a perspective view illustrating an example in which the blood pressure measurement device is attached to the wrist.

FIG. 16 is a perspective view illustrating an example in which the blood pressure measurement device is attached to the wrist.

FIG. 17 is a perspective view illustrating an example in which the blood pressure measurement device is attached to the wrist.

FIG. 18 is a cross-sectional view illustrating a configuration of a curler and a cuff structure of a blood pressure measurement device according to a second embodiment of the present invention.

FIG. 19 is a cross-sectional view illustrating a modified example of the configuration of the curler and the cuff structure of the blood pressure measurement device.

FIG. 20 is a cross-sectional view illustrating a configuration of another modified example of the curler of the blood pressure measurement device.

FIG. 21 is a cross-sectional view illustrating a configuration of another modified example of the curler of the blood pressure measurement device.

FIG. 22 is a cross-sectional view illustrating a configuration of another modified example of the curler and the cuff structure of the blood pressure measurement device.

FIG. 23 is a cross-sectional view illustrating a configuration of another modified example of the curler and the cuff structure of the blood pressure measurement device.

FIG. 24 is a perspective view illustrating a configuration of a blood pressure measurement device according to a third embodiment of the present invention.

FIG. 25 is a cross-sectional view illustrating the configuration of the blood pressure measurement device.

FIG. 26 is a block diagram illustrating the configuration of the blood pressure measurement device.

### DESCRIPTION OF EMBODIMENTS

### First Embodiment

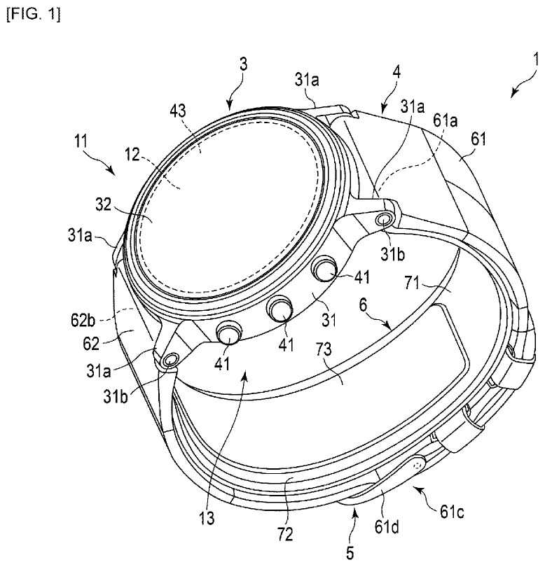

An example of a blood pressure measurement device 1 according to a first embodiment of the present invention will be described below with reference to FIGS. 1 to 13.

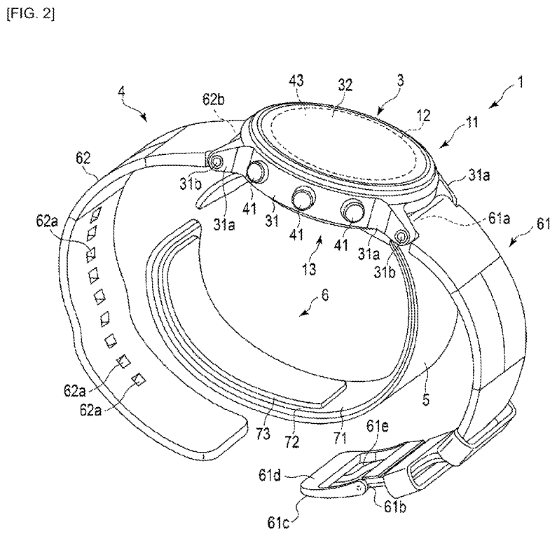

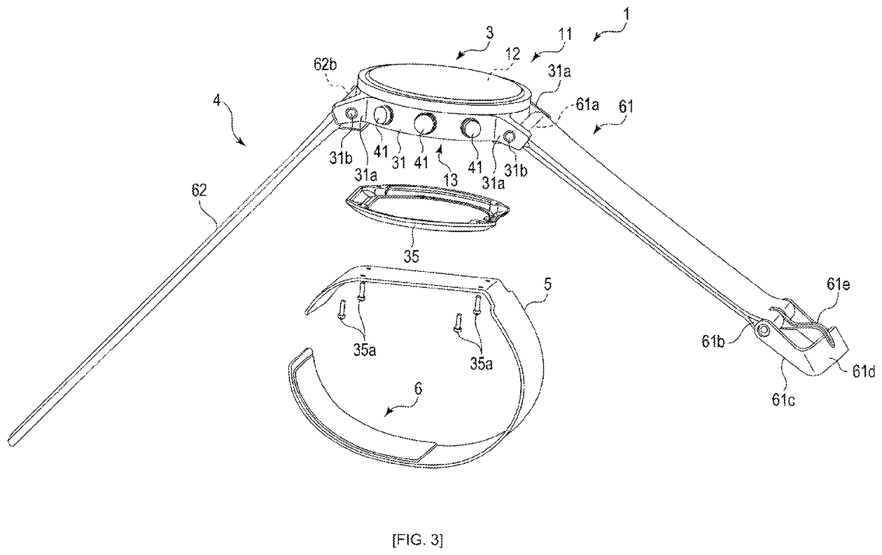

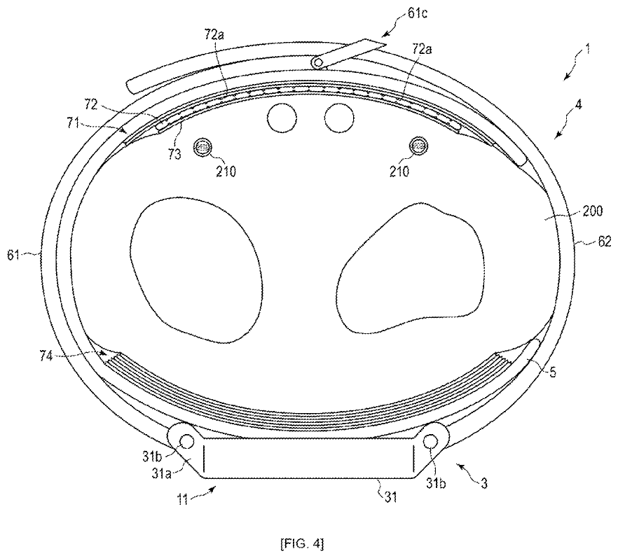

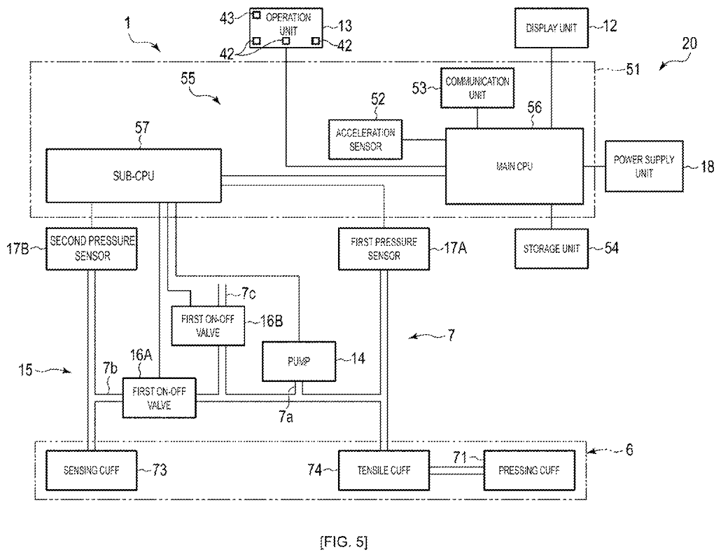

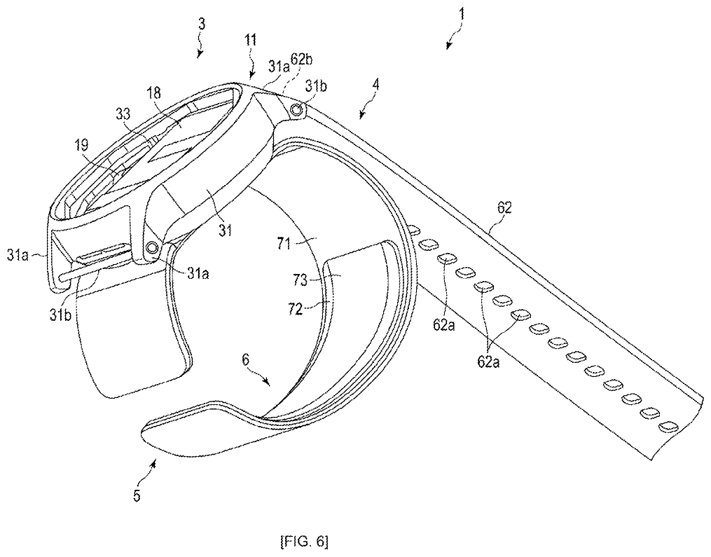

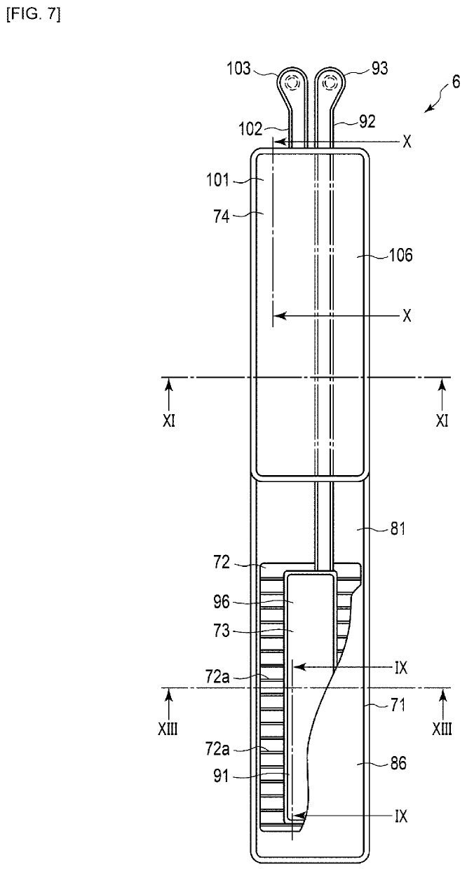

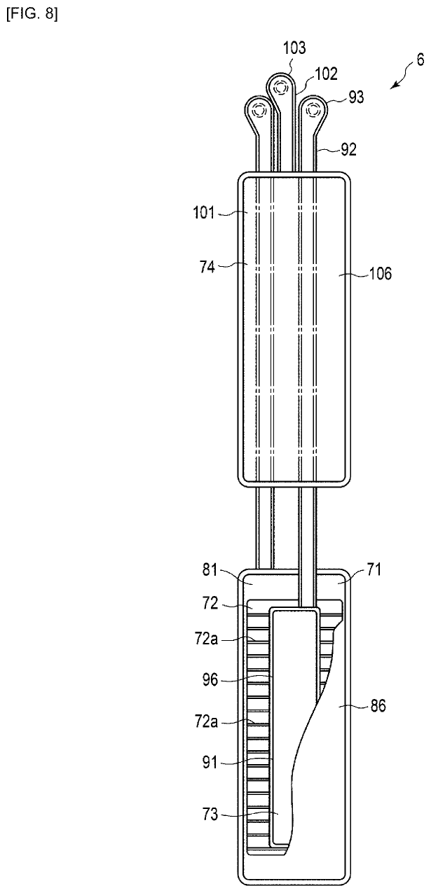

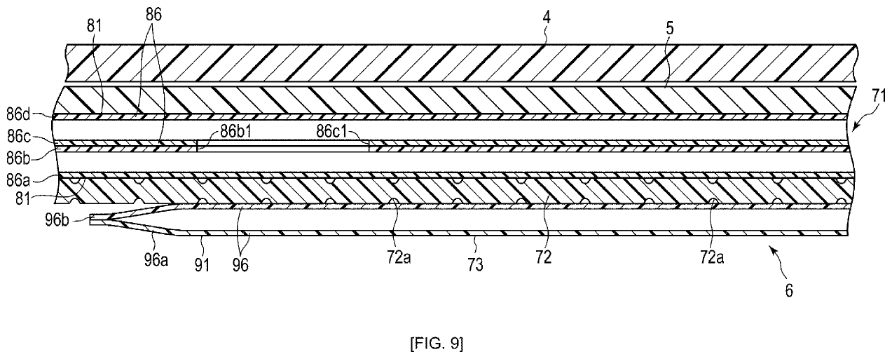

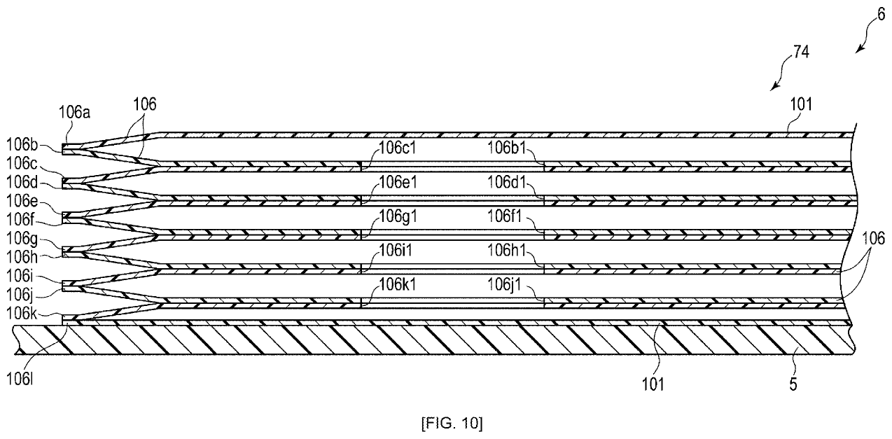

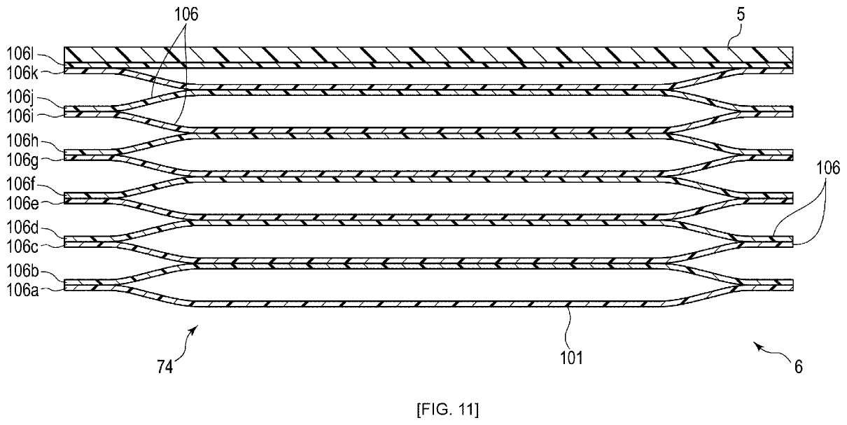

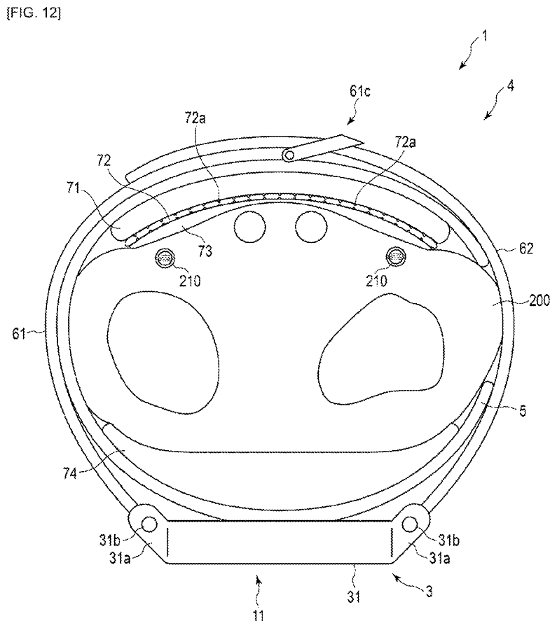

FIG. 1 is a perspective view illustrating a configuration of the blood pressure measurement device 1 according to an embodiment of the present invention in a state in which a belt 4 is closed. FIG. 2 is a perspective view illustrating the configuration of the blood pressure measurement device 1 in a state in which the belt 4 is open. FIG. 3 is an exploded perspective view illustrating the configuration of the blood pressure measurement device 1. FIG. 4 is an explanatory diagram illustrating, in cross section, a state in which the blood pressure measurement device 1 is attached to the wrist 200. FIG. 5 is a block diagram illustrating the configuration of the blood pressure measurement device 1. FIG. 6 is a perspective view illustrating a configuration of a device body 3 and a curler 5 of the blood pressure measurement device 1. FIG. 7 is a plan view illustrating a configuration of a cuff structure 6 of the blood pressure measurement device 1. FIG. 8 is a plan view illustrating another configuration of the cuff structure 6 of the blood pressure measurement device 1. FIG. 9 is a cross-sectional view illustrating a configuration of the belt 4, the curler 5, and the cuff structure 6 on a palm-side cuff 71 side of the blood pressure measurement device 1, which is taken along line IX-IX in FIG. 7. FIG. 10 is a cross-sectional view illustrating a configuration of the curler 5 and the cuff structure 6 on a back-side cuff 74 side of the blood pressure measurement device 1, which is taken along line X-X in FIG. 7. FIG. 11 is a cross-sectional view illustrating a configuration of the cuff structure 6 with the curler 5 and a tube 92 omitted, on the back-side cuff 74 side of the blood pressure measurement device 1, which is taken along line XI-XI in FIG. 7. FIG. 12 is an explanatory diagram illustrating the configuration in which the cuff structure 6 is inflated in a state in which the blood pressure measurement device 1 is attached to the wrist 200. FIG. 13 is a cross-sectional view illustrating the configuration in which the cuff structure 6 is inflated in a state in which the blood pressure measurement device 1 is attached to the wrist, which is taken along line XIII-XIII in FIG. 7.

The blood pressure measurement device 1 is an electronic blood pressure measurement device attached to a living body. The present embodiment will be described using an electronic blood pressure measurement device having an aspect of a wearable device attached to a wrist 200 of the living body.

As illustrated in FIGS. 1 to 3, the blood pressure measurement device 1 includes a device body 3, a belt 4 that fixes the device body 3 at the wrist, a curler 5 disposed between the belt 4 and the wrist, a cuff structure 6 including a palm-side cuff 71, a sensing cuff 73, and a back-side cuff 74, and a fluid circuit 7 fluidly connecting the device body 3 and the cuff structure 6.

As illustrated in FIGS. 1 to 5, the device body 3 includes, for example, a case 11, a display unit 12, an operation unit 13, a pump 14, a flow path unit 15, an on-off valve 16, a pressure sensor 17, a power supply unit 18, a vibration motor 19, and a control substrate 20. The device body 3 feeds a fluid to the cuff structure 6 using the pump 14, the on-off valve 16, the pressure sensor 17, the control substrate 20, and the like.

As illustrated in FIGS. 1 to 3, the case 11 includes an outer case 31, a windshield 32 that covers an upper opening of the outer case 31, a base 33 provided at a lower portion of an interior of the outer case 31, and a back lid 35 covering a lower portion of the outer case 31.

The outer case 31 is formed in a cylindrical shape. The outer case 31 includes pairs of lugs 31a provided at respective symmetrical positions in the circumferential direction of an outer circumferential surface, and spring rods 31b each provided between the lugs 31 of each of the two pairs of lugs 31a. The windshield 32 is, for example, a circular glass plate.

The base portion 33 holds the display unit 12, the operation unit 13, the pump 14, the on-off valve 16, the pressure sensor 17, the power supply unit 18, the vibration motor 19, and the control substrate 20. Additionally, the base 33 constitutes a portion of the flow path unit 15 that makes the pump 14 and the cuff structure 6 fluidly continuous.

The back lid 35 covers a living body side end portion of the outer case 31. The back lid 35 is fixed to the living body side end portion of the outer case 31 or the base 33 using, for example, four screws 35a or the like.

The display unit 12 is disposed on the base portion 33 of the outer case 31 and directly below the windshield 32. As illustrated in FIG. 5, the display unit 12 is electrically connected to the control substrate 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, the operation unit 13 includes a plurality of buttons 41 provided on the case 11, a sensor 42 that detects operation of the buttons 41, and a touch panel 43 provided on the display unit 12 or the windshield 32, as illustrated in FIG. 5. When operated by the user, the operation unit 13 converts an instruction into an electrical signal. The sensor 42 and the touch panel 43 are electrically connected to the control substrate 20 to output electrical signals to the control substrate 20.

As the plurality of buttons 41, for example, three buttons are provided. The buttons 41 are supported by the base 33 and protrude from the outer circumferential surface of the outer case 31. The plurality of buttons 41 and a plurality of the sensors 42 are supported by the base 33. The touch panel 43 is integrally provided on the windshield 32, for example.

The pump 14 is, for example, a piezoelectric pump. The pump 14 compresses air and feeds compressed air to the cuff structure 6 through the flow path unit 15. The pump 14 is electrically connected to the control substrate 20.

The flow path unit 15 constitutes a flow path connecting from the pump 14 to the palm-side cuff 71 and the back-side cuff 74 and a flow path connecting from the pump 14 to the sensing cuff 73, as illustrated in FIG. 5. Additionally, the flow path unit 15 constitutes a flow path connecting from the palm-side cuff 71 and the back-side cuff 74 to the atmosphere, and a flow path connecting from the sensing cuff 73 to the atmosphere. The flow path unit 15 is a flow path of air constituted by a hollow portion, a groove, a tube, or the like provided in the base portion 33 and the like.

The on-off valve 16 opens and closes a portion of the flow path 15. A plurality of the on-off valves 16 is provided, for example, as illustrated in FIG. 5, and selectively opens and closes the flow path connecting from the pump 14 to the palm-side cuff 71 and the back-side cuff 74, the flow path connecting from the pump 14 to the sensing cuff 73, the flow path connecting from the palm-side cuff 71 and the back-side cuff 74 to the atmosphere, and the flow path connecting from the sensing cuff 73 to the atmosphere, by the combination of opening and closing of each of the on-off valves 16. For example, two on-off valves 16 are used.

The pressure sensor 17 detects the pressures in the palm-side cuff 71, the sensing cuff 73 and the back-side cuff 74. The pressure sensor 17 is electrically connected to the control substrate 20. The pressure sensor 17 converts a detected pressure into an electrical signal, and outputs the electrical signal to the control substrate 20. The pressure sensor 17 is provided in the flow path connecting from the pump 14 to the palm-side cuff 71 and the back-side cuff 74 and in the flow path connecting from the pump 14 to the sensing cuff 73, as illustrated in FIG. 5. These flow paths are continuous through the palm-side cuff 71, the sensing cuff 73, and the back-side cuff 74, and thus the pressure in these flow paths corresponds to the pressure in the internal space of the palm-side cuff 71, the sensing cuff 73, and the back-side cuff 74.

The power supply unit 18 is, for example, a secondary battery such as a lithium ion battery. The power supply unit 18 is electrically connected to the control substrate 20. The power supply unit 18 supplies power to the control substrate 20.

As illustrated in FIGS. 5 and 6, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base 33 of the case 11 using screws or the like.

The acceleration sensor 52 is, for example, a 3-axis acceleration sensor. The acceleration sensor 52 outputs, to the control unit 55, an acceleration signal representing acceleration of the device body 3 in three directions orthogonal to one another. For example, the acceleration sensor 52 is used to measure, from the detected acceleration, the amount of activity of a living body to which the blood pressure measurement device 1 is attached.

The communication unit 53 is configured to be able to transmit and receive information to and from an external device wirelessly or by wire. For example, the communication unit 53 transmits information controlled by the control unit 55 and information of a measured blood pressure value, a pulse, and the like to an external device via a network, and receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, or the like.

The storage unit 54 pre-stores program data for controlling the overall blood pressure measurement device 1 and a fluid circuit 7, settings data for setting various functions of the blood pressure measurement device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measurement device 1 and operation of the fluid circuit. The control unit 55 is electrically connected to and supplies power to the display unit 12, the operation unit 13, the pump 14, each of the on-off valves 16 and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 5, the control unit 55 includes a main Central Processing Unit (CPU) 56 that controls operation of the overall blood pressure measurement device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensor 17, and outputs an image signal corresponding to the measurement results to the display unit 12.

For example, the sub-CPU 57 drives the pump 14 and the on-off valves 16 to feed compressed air to the palm-side cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signal output by the pressure sensors 17. The sub-CPU 57 controls the pump 14 and the on-off valves 16 to selectively feed compressed air to the palm-side cuff 71 and the sensing cuff 73 and selectively depressurize the palm-side cuff 71 and the sensing cuff 73.

As illustrated in FIGS. 1 to 3, the belt 4 includes a first belt 61 provided on a first pair of lugs 31a and a first spring rod 31b, and a second belt 62 provided on a second pair of lugs 31a and a second spring rod 31b. The belt 4 is wrapped around the wrist 200 with a curler 5 in between.

The first belt 61 is referred to as a so-called a parent and is configured like a band. The first belt 61 includes a first hole portion 61a provided at a first end portion of the first belt 61 and extending orthogonally to the longitudinal direction of the first belt 61, a second hole portion 61b provided at a second end portion of the first belt 61 and extending orthogonally to the longitudinal direction of the first belt 61, and a buckle 61c provided on the second hole portion 61b. The first hole portion 61a has an inner diameter at which the spring rod 31b can be inserted into the first hole portion 61a and at which the first belt 61 can rotate with respect to the spring rod 31b. In other words, the first belt 61 is rotatably held by the outer case 31 by disposing the first hole portion 61a between the pair of lugs 31a and around the spring rod 31b.

The second hole portion 61b is provided at a tip of the first belt 61. The buckle 61c includes a frame body 61d in a rectangular frame shape and a prong 61e rotatably attached to the frame body 61d. A side of the frame body 61d to which the prong 61e is attached is inserted into the second hole portion 61b. The frame body 61d is attached to the first belt 61 with the prong 61e in between such that the frame body 61d is rotatable with respect to the first belt 61.

The second belt 62 is referred to as a so-called blade tip, and is configured in a band-like shape having a width at which the second belt 62 can be inserted into the frame body 61d. In addition, the second belt 62 includes a plurality of small holes 62a into which the prong 61e is inserted. Additionally, the second belt 62 includes a third hole portion 62b provided at first end portion of the second belt 62 and extending orthogonally to the longitudinal direction of the second belt 62. The third hole portion 62b has an inner diameter at which the spring rod 31b can be inserted into the third hole portion 62b and at which the second belt 62 can rotate with respect to the spring rod 31b. In other words, the second belt 62 is rotatably held by the outer case 31 by disposing the third hole portion 62b between the pair of lugs 31a and around the spring rod 31b.

In the belt 4 as described above, the second belt 62 is inserted into the frame body 61d, and the prong 61e is inserted into the small hole 62a. Thus, the first belt 61 and the second belt 62 of the belt 4 are integrally connected together, and then the belt 4 comes to have an annular shape following along the circumferential direction of the wrist 200 along with the outer case 31.

As illustrated in FIG. 4, the curler 5 is configured in a band-like shape that curves in such a manner as to follow along the circumferential direction of the wrist. The curler 5 is formed with a first end and a second end spaced apart from each other.

For example, a first end-side outer surface of the curler 5 is fixed to the back lid 35 of the device body 3. The first end and the second end of the curler 5 are disposed at positions where the first end and the second end protrude from the back lid 35. Furthermore, the first end and the second end of the curler 5 are located adjacent to each other at a predetermined distance from each other.

As a specific example, the curler 5 is fixed to a living body side end portion of the outer case 31 or the base 33 along with the back lid 35 using screws 35a or the like. Additionally, the curler 5 is fixed to the back lid 35 such that the first end and the second end are located on one lateral side of the wrist 200 when the blood pressure measurement device 1 is attached to the wrist 200.

As a specific example, as illustrated in FIG. 1, FIG. 2, and FIG. 4, the curler 5 has a shape that curves along a direction orthogonal to the circumferential direction of the wrist, in other words, along the circumferential direction of the wrist 200 in a side view from the longitudinal direction of the wrist. The curler 5 extends, for example, from the device body 3 through the hand back side of the wrist 200 and one lateral side of the wrist 200 to the hand palm side of the wrist 200 and toward the other lateral side of the wrist 200. Specifically, by curving along the circumferential direction of the wrist 200, the curler 5 is disposed across the most of the wrist 200 in the circumferential direction, with both ends of the curler 5 spaced at a predetermined distance from each other.

The curler 5 has hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist.

The cuff structure 6 is disposed on an inner circumferential surface of the curler 5, and is held along the shape of the inner circumferential surface of the curler 5. As a specific example, the cuff structure 6 is fixed to the curler 5 by disposing the palm-side cuff 71 and the back-side cuff 74 on the inner circumferential surface of the curler 5, and thermally welding the palm-side cuff 71 and the back-side cuff 74 to an outer circumferential surface or the inner circumferential surface of the curler 5. In the present embodiment, the palm-side cuff 71 and the back-side cuff 74 are thermally welded to the inner circumferential surface of the curler 5.

The curler 5 is formed of a thermoplastic resin material. Furthermore, a material that is harder than the palm-side cuff 71 and the back-side cuff 74 is used for the curler 5. For example, the curler 5 is constituted by a single material. For example, the resin material constituting the curler 5 includes a material similar to the resin material constituting the palm-side cuff 71 and the back-side cuff 74.

Specifically, the resin material constituting the curler 5 is constituted by a material that is compatible in welding with the resin material constituting the palm-side cuff 71 and the back-side cuff 74. Here, “compatibility” refers to the degree of mixing of the resin materials softened or melted during welding, and “high compatibility” means that junction can be achieved in which the resin materials softened or melted during welding mix together at a suitable degree, that is, junction can be achieved at a required junction strength. Specifically, the compatible resin materials refer to, in the present embodiment, two resin materials, in which the resin material constituting the curler 5 and the resin material constituting the palm-side cuff 71 and the back-side cuff 74 suitably mix together during thermal welding, and the resin material of the curler 5 and the resin material of the palm-side cuff 71 and the back-side cuff 74 can be integrated together at the welding portion after the welding.

In addition, the resin material constituting the curler 5 is constituted by a material having a softening temperature identical or close to a softening temperature of the resin material constituting the palm-side cuff 71 and the back-side cuff 74. Note that the softening temperatures of the resin materials constituting the curler 5, the palm-side cuff 71, and the back-side cuff 74 can be set as appropriate as long as the resin materials are softened and melted together at these temperatures when the curler 5 and the cuff structure 6 are welded to each other. For example, as a welding method for the curler 5, the palm-side cuff 71, and the back-side cuff 74, welder welding, laser welding, thermal welding, hot air welding, induction welding, ultrasonic welding, and radiant welding can be used.

Examples of thermoplastic resin material constituting the curler 5 may include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin. The curler 5 is formed, for example, to a thickness of approximately 1 mm.

As illustrated in FIGS. 1 to 4 and 7 to 13, the cuff structure 6 includes the palm-side cuff (cuff) 71, a back plate 72, the sensing cuff 73, and the back-side cuff (cuff) 74. The cuff structure 6 is fixed to the curler 5. The cuff structure 6 includes the palm-side cuff 71, the back plate 72, and the sensing cuff 73 that are stacked one another and disposed on the curler 5, and the back-side cuff 74 that is spaced apart from the palm-side cuff 71, the back plate 72, and the sensing cuff 73 and disposed on the curler 5.

As a specific example, the cuff structure 6 includes the palm-side cuff 71, the back plate 72, the sensing cuff 73, and the back-side cuff 74 that are disposed on an inner surface of the curler 5. The cuff structure 6 is fixed to the inner surface of the curler 5 on the hand palm side of the wrist 200 with the palm-side cuff 71, the back plate 72, and the sensing cuff 73 stacked in this order from the inner surface of the curler 5 toward the living body. In addition, the cuff structure 6 includes the back-side cuff 74 disposed on the inner surface of the curler 5 on the hand back side of the wrist 200. Each of the members of the cuff structure 6 is fixed to an adjacent member of the cuff structure 6 in a stacking direction with a double-sided tape, an adhesive, or the like.

The palm-side cuff 71 is a so-called pressing cuff. The palm-side cuff 71 is fluidly connected to the pump 14 through the flow path unit 15. The palm-side cuff 71 is inflated to press the back plate 72 and the sensing cuff 73 toward the living body side. The palm-side cuff 71 includes air bags 81 in a plurality of, for example, two layers. The palm-side cuff 71 is constituted by a resin material that is similar to the resin material of the curler 5, which is highly compatible with the resin material of the curler 5 when the palm-side cuff 71 is thermally welded to the curler 5.

Here, the air bags 81 are bag-like structures, and in the present embodiment, the blood pressure measurement device 1 is configured to use air with the pump 14, and thus the present embodiment will be described using the air bags. However, in a case where a fluid other than air is used, the bag-like structures may be fluid bags such as liquid bags. The plurality of air bags 81 are stacked and are in fluid communication with one another in the stacking direction.

Each of the air bags 81 is constituted in a rectangular shape that is long in one direction. The air bag 81 is constituted, for example, by combining two sheet members 86 that are long in one direction, and thermally welding edges of the sheet members. As a specific example, as illustrated in FIGS. 7 to 9, the two-layer air bags 81 include a first sheet member 86a, a second sheet member 86b, a third sheet member 86c, and a fourth sheet member 86d in this order from the living body side. The second sheet member 86b constitutes a first-layer air bag 81 along with the first sheet member 86a, the third sheet member 86c is integrally bonded to the second sheet member 86b, and the fourth sheet member 86d constitutes a second-layer air bag 81 along with the third sheet member 86c. Note that the two-layer air bags 81 are integrally constituted by joining each of the sheet members 86 of the adjacent air bags 81 by bonding with a double-sided tape, an adhesive, or the like, or welding or the like.

Edge portions of four sides of the first sheet member 86a are welded to corresponding edge portions of four sides of the second sheet member 86b to constitute the air bag 81. The second sheet member 86b and the third sheet member 86c are disposed facing each other, and each includes a plurality of openings 86b1 and 86c1 through which the two air bags 81 are fluidly continuous. The fourth sheet member 86d is disposed on the curler 5 and is thermally welded to the inner circumferential surface or the outer circumferential surface of the curler 5.

Edge portions of four sides of the third sheet member 86c are welded to corresponding edge portions of four sides of the fourth sheet member 86d to constitute the air bag 81.

The back plate 72 is applied to an outer surface of the first sheet member 86a of the palm-side cuff 71 with an adhesive layer, a double-sided tape, or the like. The back plate 72 is formed in a plate shape using a resin material. The back plate 72 is made of polypropylene, for example, and is formed into a plate shape having a thickness of approximately 1 mm. The back plate 72 has shape followability.

Here, “shape followability” refers to a function of the backplate 72 by which the back plate 72 can be deformed in such a manner as to follow the shape of a contacted portion of the wrist 200 to be disposed, the contacted portion of the wrist 200 refers to a region of the wrist 200 that is faced by the back plate 72, and the contact as used herein includes both direct contact and indirect contact with the sensing cuff 73 in between.

For example, as illustrated in FIG. 9, the back plate 72 includes a plurality of grooves 72a formed in both main surfaces of the back plate 72 and extending in a direction orthogonal to the longitudinal direction. As illustrated in FIG. 9, a plurality of the grooves 72a are provided in both main surfaces of the back plate 72. The plurality of grooves 72a provided in one of the main surfaces face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a, and thus the portions including the plurality of grooves 72a are easily deformed. Thus, the back plate 72 is deformed in such a manner as to follow to the shape of the wrist 200, and has shape followability of extending in the circumferential direction of the wrist. The back plate 72 is formed such that the length of the back plate 72 is sufficient to cover the hand palm side of the wrist 200. The back plate 72 transfers the pressing force from the palm-side cuff 71 to the back plate 72 side main surface of the sensing cuff 73 in a state in which the back plate 72 is extending along the shape of the wrist 200.

The sensing cuff 73 is fixed to the living body side main surface of the back plate 72. The sensing cuff 73 is in direct contact with a region of the wrist 200 where an artery 210 resides, as illustrated in FIGS. 12 and 13. The artery 210 as used herein is the radial artery and the ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm-side region of the wrist 200 in which the artery 210 resides. The sensing cuff 73 is pressed by the inflated palm-side cuff 71 toward the living body side with the back plate 72 in between.

As a specific example, the sensing cuff 73 includes one air bag 91, a tube 92 that communicates with the air bag 91, and a connection portion 93 provided at a tip of the tube 92. One main surface of the air bag 91 of the sensing cuff 73 is fixed to the back plate 72. For example, the sensing cuff 73 is applied to the living body side main surface of the back plate 72 using a double-sided tape, an adhesive layer, or the like.

Here, the air bag 91 is a bag-like structure, and in the present embodiment, the blood pressure measurement device 1 is configured to use air with the pump 14, and thus the present embodiment will be described using the air bag. However, in a case where a fluid other than air is used, the bag-like structure may be a liquid bag and the like.

The air bag 91 is constituted in a rectangular shape that is long in one direction. The air bag 91 is constituted, for example, by combining two sheet members 96 that are long in one direction, and thermally welding edges of the sheet members. As a specific example, the air bag 91 includes a fifth sheet member 96a and a sixth sheet member 96b in this order from the living body side as illustrated in FIGS. 9 and 13.

For example, the fifth sheet member 96a and the sixth sheet member 96b are fixed by welding, with a tube 92 that is fluidly continuous with the internal space of the air bag 91 being disposed on one side of each of the fifth sheet member 96a and the sixth sheet member 96b. For example, the fifth sheet member 96a and the sixth sheet member 96b are welded together integrally with the tube 92 by welding edge portions of four sides of the fifth sheet member 96a to corresponding edge portions of four sides of the sixth sheet member 96b in a state in which the tube 92 is disposed between the fifth sheet member 96a and the sixth sheet member 96b.

The tube 92 is provided at one longitudinal end portion of the air bag 91. As a specific example, the tube 92 is provided at an end portion of the air bag 91 near the device body 3. The tube 92 includes the connection portion 93 at the tip. The tube 92 is connected to the flow path unit 15 and constitutes a flow path between the device body 3 and the air bag 91. The connection portion 93 is connected to the flow path unit 15. The connection portion 93 is, for example, a nipple.

The back-side cuff 74 is a so-called tensile cuff. The back-side cuff 74 is fluidly connected to the pump 14 through the flow path unit 15. The back-side cuff 74 is inflated to press the curler 5 such that the curler 5 is spaced apart from the wrist 200, pulling the belt 4 and the curler 5 toward the hand back side of the wrist 200. The back-side cuff 74 includes air bags 101 including a plurality of, for example, six layers, a tube 102 in communication with the air bags 101, and a connection portion 103 provided at a tip of the tube 102.

Additionally, the back-side cuff 74 is configured such that the thickness of the back-side cuff 74 in an inflating direction, in the present embodiment, in the direction in which the curler 5 and the wrist 200 face each other, during inflation, is larger than the thickness of the palm-side cuff 71 in the inflating direction during inflation and the thickness of the sensing cuff 73 in the inflating direction during inflation. Specifically, the air bags 101 of the back-side cuff 74 include more layers than the air bags 81 in the palm-side cuff 71 and the air bag 91 in the sensing cuff 73, and are thicker than the palm-side cuff 71 and the sensing cuff 73 when the air bags 101 are inflated from the curler 5 toward the wrist 200.

Here, the air bag 101 is a bag-like structure, and in the present embodiment, the blood pressure measurement device 1 is configured to use air with the pump 14, and thus the present embodiment will be described using the air bag. However, in a case where a fluid other than air is used, the bag-like structure may be a fluid bag such as a liquid bag. A plurality of the air bags 101 are stacked and are in fluid communication in the stacking direction.

The air bag 101 is constituted in a rectangular shape that is long in one direction. The air bag 101 is constituted, for example, by combining two sheet members 106 that are long in one direction, and thermally welding edges of the sheet members. As a specific example, as illustrated in FIGS. 10 and 11, the six-layer air bags 101 include a seventh sheet member 106a, an eighth sheet member 106b, a ninth sheet member 106c, a tenth sheet member 106d, an eleventh sheet member 106e, a twelfth sheet member 106f, a thirteenth sheet member 106 g, a fourteenth sheet member 106h, a fifteenth sheet member 106i, a sixteenth sheet member 106j, a seventeenth sheet member 106k, and an eighteenth sheet member 106 l in this order from the living body side. Note that the six-layer air bags 101 are integrally constituted by joining each of the sheet members 106 of the adjacent air bags 101 by bonding with a double-sided tape, an adhesive, or the like, or welding or the like.

Edge portions of four sides of the seventh sheet member 106a are welded to corresponding edge portions of four sides of the eighth sheet member 106b to constitute a first-layer air bag 101. The eighth sheet member 106b and the ninth sheet member 106c are disposed facing each other and are integrally bonded together. The eighth sheet member 106b and the ninth sheet member 106c include a plurality of openings 106b1 and 106c1 through which the adjacent air bags 101 are fluidly continuous. Edge portions of four sides of the ninth sheet member 106c are welded to corresponding edge portions of four sides of the tenth sheet member 106d to constitute a second-layer air bag 101.

The tenth sheet member 106d and the eleventh sheet member 106e are disposed facing each other and are integrally bonded together. The tenth sheet member 106d and the eleventh sheet member 106e include a plurality of openings 106d1 and 106e1 through which the adjacent air bags 101 are fluidly continuous. Edge portions of four sides of the eleventh sheet member 106e are welded to corresponding edge portions of four sides of the twelfth sheet member 106f to constitute a third-layer air bag 101.

The twelfth sheet member 106f and the thirteenth sheet member 106 g are disposed facing each other and are integrally bonded together. The twelfth sheet member 106f and the thirteenth sheet member 106 g include a plurality of openings 106f1 and 106g1 through which the adjacent air bags 101 are fluidly continuous. Edge portions of four sides of the thirteenth sheet member 106 g are welded to corresponding edge portions of four sides of the fourteenth sheet member 106 h to constitute a fourth-layer air bag 101.

The fourteenth sheet member 106 h and the fifteenth sheet member 106i are disposed facing each other and are integrally bonded together. The fourteenth sheet member 106 h and the fifteenth sheet member 106i include a plurality of openings 106h1 and 106i1 through which the adjacent air bags 101 are fluidly continuous. Edge portions of four sides of the fifteenth sheet member 106i are welded to corresponding edge portions of four sides of the sixteenth sheet member 106j to constitute a fifth-layer air bag 101.

The sixteenth sheet member 106j and the seventeenth sheet member 106k are disposed facing each other and are integrally bonded together. The sixteenth sheet member 106j and the seventeenth sheet member 106k include a plurality of openings 106j1 and 106k1 through which the adjacent air bags 101 are fluidly continuous. Edge portions of four sides of the seventeenth sheet member 106k are welded to corresponding edge portions of four sides of the eighteenth sheet member 106 l to constitute a sixth-layer air bag 101. In addition, for example, a tube 102 that is fluidly continuous with the internal space of the air bag 101 is disposed on one side of the seventeenth sheet member 106k and the eighteenth sheet member 106l, and is fixed by welding. For example, in a state in which the tube 102 is disposed between the seventeenth sheet member 106k and the eighteenth sheet member 106l, the edge portions of the seventeenth sheet member 106k are welded to the edge portions of the eighteenth sheet member 106 l in a rectangular frame shape to form the air bag 101. Thus, the tube 102 is integrally welded to the air bag 101.

For example, the sixth-layer air bag 101 as described above is constituted integrally with the second layer air bag 81 of the palm-side cuff 71. Specifically, the seventeenth sheet member 106k is constituted integrally with the third sheet member 86c, and the eighteenth sheet member 106 l is constituted integrally with the fourth sheet member 86d.

In more detail, the third sheet member 86c and the seventeenth sheet member 106k constitute a rectangular sheet member that is long in one direction, and the eighteenth sheet member 106 l and the fourth sheet member 86d constitute a rectangular sheet member that is long in one direction. Then, these sheet members are stacked one another, and welding is performed such that first end portion side is welded in a rectangular frame shape, whereas a part of one side on the second end portion side is not welded. Thus, the second-layer air bag 81 of the palm-side cuff 71 is constituted. Then, welding is performed such that the second end portion side is welded in a rectangular frame shape, whereas a part of one side on the first end portion side is not welded. Thus, the sixth-layer air bag 101 in the back-side cuff 74 is constituted. In addition, a part of one side on the facing side of each of the second-layer air bag 81 and the sixth-layer air bag 101 is not welded, and thus the second-layer air bag 81 and the sixth-layer air bag 101 are fluidly continuous.

The tube 102 is connected to one air bag 101 of the six-layer air bags 101 and is provided at one longitudinal end portion of the air bag 101. As a specific example, the tube 102 is provided on the curler 5 side of the six-layer air bags 101 and is provided at the end portion close to the device body 3. The tube 102 includes a connection portion 103 at the tip. The tube 102 constitutes a flow path included in the fluid circuit 7 and located between the device body 3 and the air bags 101. The connection portion 103 is, for example, a nipple.

Note that, as described above, in the present embodiment, the configuration has been described in which a part of the back-side cuff 74 is constituted integrally with the palm-side cuff 71 and is fluidly continuous with the palm-side cuff 71. However, no such limitation is intended. For example, as illustrated in FIG. 8, the back-side cuff 74 may be constituted separately from the palm-side cuff 71 and may be fluidly discontinuous with the palm-side cuff 71. For such a configuration, the palm-side cuff 71 may be configured such that, like the sensing cuff 73 and the back-side cuff 74, the palm-side cuff 71 is further provided with a tube and a connection portion, and in the fluid circuit 7 as well, the palm-side cuff 71 is connected to a flow path through which the fluid is fed to the palm-side cuff 71, a check valve, and a pressure sensor.

Additionally, each of the sheet members 86, 96, and 106 forming the palm-side cuff 71, the sensing cuff 73, and the back-side cuff 74 are formed of a thermoplastic resin material. The thermoplastic resin material is a thermoplastic elastomer. Examples of thermoplastic resin material constituting the sheet members 86, 96, and 106 include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin. Note that, in the palm-side cuff 71 and the sensing cuff 73, of at least the plurality of sheet members 86 and 106 constituting the air bags 81 and 101, at least the sheet members 86 and 106 welded to the curler 5 are constituted by a material similar to the material of the curler 5.

For example, the sheet members 86, 96, and 106 are formed using a molding method such as T-die extrusion molding or injection molding. After being molded by each molding method, the sheet members 86, 96, and 106 are sized into predetermined shapes, and the sized individual pieces are joined by welding or the like to constitute bag-like structures 81, 91, and 101. A high frequency welder or laser welding is used as the welding method.

Now, an example of the resin material used in the curler 5 and the cuff structure 6 will be described. First, as described above, the curler 5 is required to have a hardness appropriate to provide flexibility and shape retainability.

Additionally, the cuff structure 6 is configured such that the air bags 81, 91, and 101 are inflated, the air bag 81 is constituted by welding the sheet members 86, 96, and 106 together, and the air bag 81 in the palm-side cuff 71 and the air bag 101 in the back-side cuff 74 are welded to the curler 5.

For this reason, at least the curler 5 is compatible, during welding, with at least the sheet members 86 of the air bag 81 in the palm-side cuff 71 and the sheet members 106 of the air bag 101 in the back-side cuff 74 that are welded to the curler 5. The curler 5 and the sheet members 86 and 106 are constituted by similar materials in order to be in a suitable combination of softening temperatures.

Note that it is sufficient that, in the palm-side cuff 71 and the back-side cuff 74, the sheet members 86 and 106 welded to the curler 5 are constituted by a material similar to the material of the curler 5. However, the adjacent sheet members 86 and 106 are welded that are stacked when the air bags 81 and 101 are formed, all the sheet members 86 are preferably constituted by the same material.

For example, a thermoplastic polyurethane resin (TPU) 1174D is used for the curler 5, and a thermoplastic polyurethane resin (TPU) R195A is used for the palm-side cuff 71 and the back-side cuff 74. Note that the sheet members 86, 96, and 106 may have a single layer structure or a multilayer structure, as long as the curler 5 and the palm-side cuff 71 and the back-side cuff 74 as well as the adjacent sheet members 86, 96, and 106 can be suitably welded together.

The fluid circuit 7 is constituted by the case 11, the pump 14, the flow path unit 15, the on-off valves 16, the pressure sensors 17, the palm-side cuff 71, the sensing cuff 73, and the back-side cuff 74. A specific example of the fluid circuit 7 will be described below with two on-off valves 16 that are used in the fluid circuit 7 being designated as a first on-off valve 16A and a second on-off valve 16B, and two pressure sensors 17 that are used in the fluid circuit 17 being designated as a first pressure sensor 17A and a second pressure sensor 17B.

As illustrated in FIG. 5, the fluid circuit 7 includes, for example, a first flow path 7a that makes the palm-side cuff 71 and the back-side cuff 74 continuous with the pump 14, a second flow path 7b constituted by branching from a middle portion of the first flow path 7a and making the sensing cuff 73 continuous with the pump 14, and a third flow path 7c connecting the first flow path 7a to the atmosphere. Additionally, the first flow path 7a includes the first pressure sensor 17A. The first on-off valve 16A is provided between the first flow path 7a and the second flow path 7b. The second flow path 7b includes a second pressure sensor 17B. The second on-off valve 16B is provided between the first flow path 7a and the third flow path 7c.

In the fluid circuit 7 as described above, the first on-off valve 16A and the second on-off valve 16B are closed to connect only the first flow path 7a to the pump 14, and the pump 14 and the palm-side cuff 71 are fluidly connected. In the fluid circuit 7, the first on-off valve 16A is opened and the second on-off valve 16B is closed to connect the first flow path 7a and the second flow path 7b, thus fluidly connecting the pump 14 and the back-side cuff 74, the back-side cuff 74 and the palm-side cuff 71, and the pump 14 and the sensing cuff 73. In the fluid circuit 7, the first on-off valve 16A is closed and the second on-off valve 16B is opened to connect the first flow path 7a and the third flow path 7c, fluidly connecting the palm-side cuff 71, the back-side cuff 74, and the atmosphere together. In the fluid circuit 7, the first on-off valve 16A and the second on-off valve 16B are opened to connect the first flow path 7a, the second flow path 7b, and the third flow path 7c, fluidly connecting the palm-side cuff 71, the sensing cuff 73, the back-side cuff 74, and the atmosphere together.

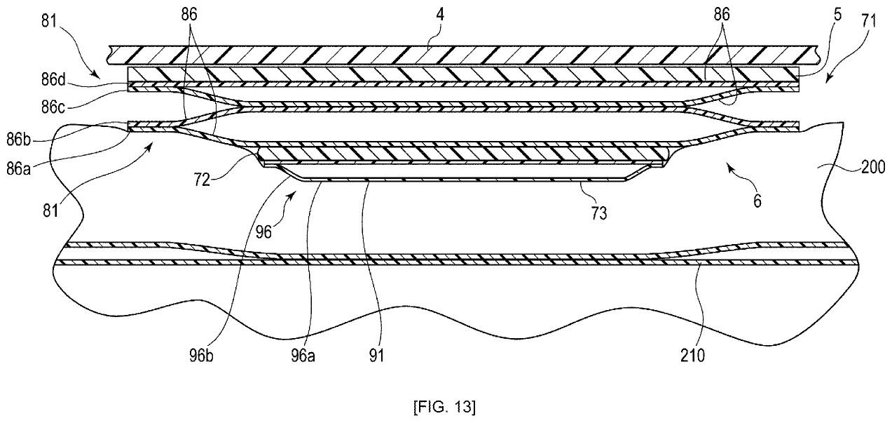

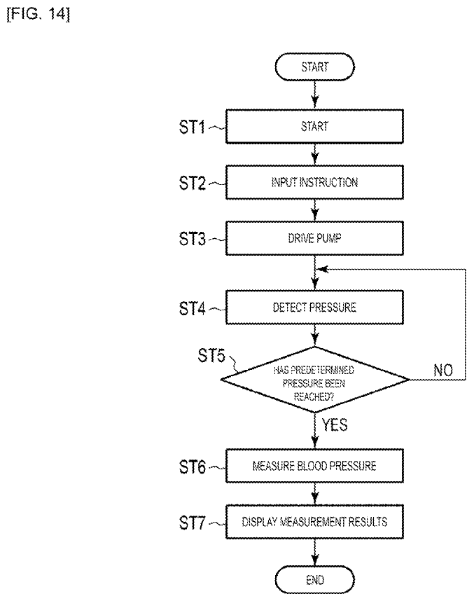

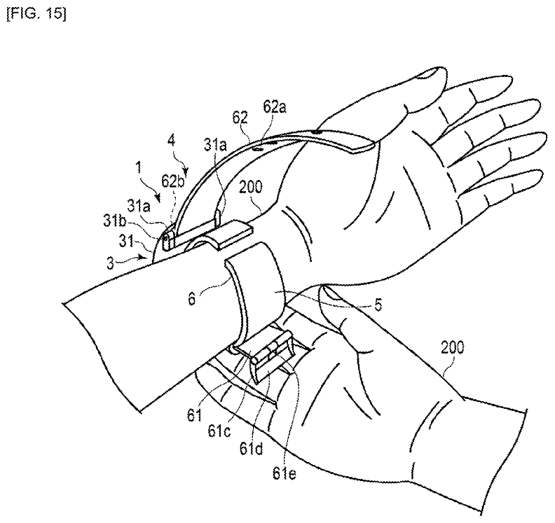

Now, an example of measurement of a blood pressure value using the blood pressure measurement device 1 will be described using FIGS. 14 to 17. FIG. 14 is a flowchart illustrating an example of a blood pressure measurement using the blood pressure measurement device 1, illustrating both an operation of a user and an operation of the control unit 55. Additionally, FIGS. 15 to 17 illustrate an example of the user wearing the blood pressure measurement device 1 on the wrist 200.

First, the user attaches the blood pressure measurement device 1 to the wrist 200 (step ST1). As a specific example, for example, the user inserts one of the wrists 200 into the curler 5, as illustrated in FIG. 15.

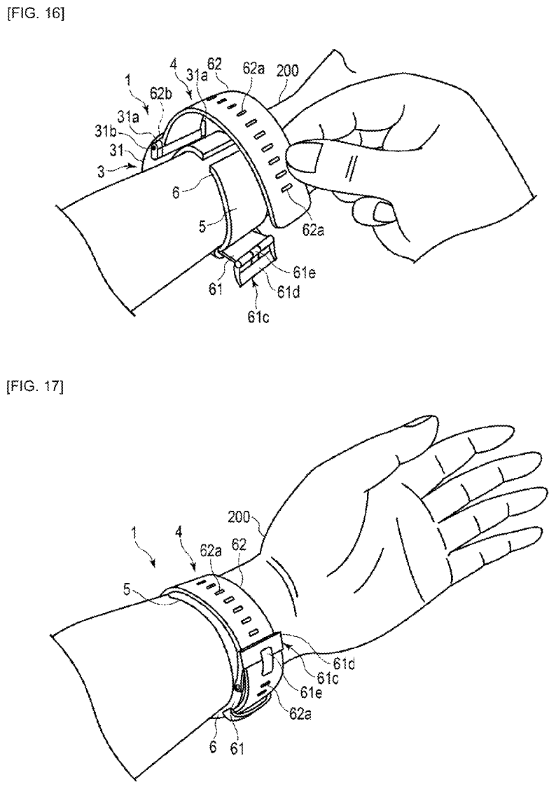

At this time, in the blood pressure measurement device 1, the device body 3 and the sensing cuff 73 are disposed at opposite positions in the curler 5, and thus the sensing cuff 73 is disposed in a region on the hand palm side of the wrist 200 in which the artery 210 resides. Thus, the device body 3 and the back-side cuff 74 are disposed on the hand back side of the wrist 200. Then, as illustrated in FIG. 16, the user passes the second belt 62 through the frame body 61d of the buckle 61c of the first belt 61 with the hand opposite to the hand on which the blood pressure measurement device 1 is disposed. The user then pulls the second belt 62 to bring the member on the inner circumferential surface side of the curler 5, that is, the cuff structure 6, into close contact with the wrist 200, and inserts the prong 61e into the small hole 62a. Thus, as illustrated in FIG. 17, the first belt 61 and the second belt 62 are connected, and the blood pressure measurement device 1 is attached to the wrist 200.

Then, the user operates the operation unit 13 to input an instruction corresponding to the start of measurement of the blood pressure value. The operation unit 13, on which an input operation of the instruction has been performed, outputs an electrical signal corresponding to the start of the measurement to the control unit 55 (step ST2). The control unit 55 receives the electrical signal, and then for example, opens the first on-off valve 16A, closes the second on-off valve 16B, and drives the pump 14 to feed compressed air to the palm-side cuff 71, the sensing cuff 73, and the back-side cuff 74 through the first flow path 7a and the second flow path 7b (step ST3). Thus, the palm-side cuff 71, the sensing cuff 73, and the back-side cuff 74 start to be inflated.

The first pressure sensor 17A and the second pressure sensor 17B detect the pressures in the palm-side cuff 71, the sensing cuff 73, and the back-side cuff 74, and outputs, to the control unit 55, electrical signals corresponding to the pressures (step ST4). Based on the received electrical signals, the control unit 55 determines whether the pressures in the internal spaces of the palm-side cuff 71, the sensing cuff 73, and the back-side cuff 74 have reached a predetermined pressure for measurement of the blood pressure (step ST5). For example, in a case where the internal pressures of the palm-side cuff 71 and the back-side cuff 74 have not reached the predetermined pressure and the internal pressure of the sensing cuff 73 has reached the predetermined pressure, the control unit 55 closes the first on-off valve 16A and feeds compressed air through the first flow path 7a.

When the internal pressures of the palm-side cuff 71 and the back-side cuff 74 and the internal pressure of the sensing cuff 73 all have reached the predetermined pressure, the control unit 55 stops driving the pump 14 (YES in step ST5). At this time, as illustrated in FIGS. 12 and 13, the palm-side cuff 71 and the back-side cuff 74 are sufficiently inflated, and the inflated palm-side cuff 71 presses the back plate 72. Additionally, the back-side cuff 74 presses against the curler 5 in a direction away from the wrist 200, and then the belt 4, the curler 5, and the device body 3 move in a direction away from the wrist 200, and as a result, the palm-side cuff 71, the back plate 72, and the sensing cuff 73 are pulled toward the wrist 200 side. In addition, when the belt 4, the curler 5, and the device body 3 move in a direction away from the wrist 200 due to the inflation of the back-side cuff 74, the belt 4 and the curler 5 move toward both lateral sides of the wrist 200, and the belt 4, the curler 5, and the device body 3 move in a state of close contact with both lateral sides of the wrist 200. Thus, the belt 4 and the curler 5, which are in close contact with the skin of the wrist 200, pull the skin on both lateral sides of the wrist 200 toward the hand back side. Note that the curler 5 may be configured to indirectly contact the skin of the wrist 200 with the sheet members 86 or 106 in between, for example, as long as the curler 5 can pull the skin of the wrist 200.

Further, the sensing cuff 73 is inflated by being fed with a predetermined amount of air such that the internal pressure equals the pressure required to measure blood pressure, and is pressed toward the wrist 200 by the back plate 72 that is pressed by the palm-side cuff 71. Thus, the sensing cuff 73 presses the artery 210 in the wrist 200 and occludes the artery 210 as illustrated in FIG. 13.

Additionally, the control unit 55, for example, controls the second on-off valve 16B and repeats the opening and closing of the second on-off valve 16B, or adjusts the degree of opening of the second on-off valve 16B to pressurize the internal space of the palm-side cuff 71. In the process of pressurization, based on the electrical signal output by the second pressure sensor 17B, the control unit 55 obtains measurement results such as blood pressure values, for example, the systolic blood pressure and the diastolic blood pressure, and the heart rate and the like (step ST6). The control unit 55 outputs an image signal corresponding to the obtained measurement results to the display unit 12, and displays the measurement results on the display unit 12 (step ST7). In addition, after the end of the blood pressure measurement, the control unit 55 opens the first on-off valve 16A and the second on-off valve 16B.

The display unit 12 receives the image signal, and then displays the measurement results on the screen. The user views the display unit 12 to confirm the measurement results. After the measurement is complete, the user removes the prong 61e from the small hole 62a, removes the second belt 62 from the frame body 61 d, and removes the wrist 200 from the curler 5, thus removing the blood pressure measurement device 1 from the wrist 200.

The blood pressure measurement device 1 according to one embodiment configured as described above, has a configuration in which the curler 5, the palm-side cuff 71, and the back-side cuff 74 are joined together by thermal welding. In addition, the blood pressure measurement device 1 has a configuration in which at least the welded regions of the curler 5, the palm-side cuff 71, and the back-side cuff 74 include thermoplastic resin materials that are compatible each other and are similar materials having the same softening temperature or similar softening temperatures.

Thus, when the curler 5 is joined to the palm-side cuff 71 and the back-side cuff 74, the palm-side cuff 71 and the back-side cuff 74 can be suitably welded to the curler 5. As a result, the junction strength of the junction portions between the curler 5 and the palm-side cuff 71 and the back-side cuff 74 can be increased. Note that “suitable welding” as used herein refers to welding in which, when a tensile load is applied to the curler 5, the palm-side cuff 71, and the back-side cuff 74 until the junction portions are separated from each other, material fracture occurs instead of interfacial peeling in the junction portions.

In this way, the junction strength between the curler 5 and, the palm-side cuff 71 and the back-side cuff 74 that are repeatedly inflated and contracted is increased, and thus the curler 5 and the cuff structure 6 have a high durability. Additionally, the cuff structure 6 is joined to the curler 5 at a high junction strength, and thus the cuff structure 6 is repeatedly inflated and contracted in an orientation along the inner circumferential surface of the curler 5. This suppresses wrinkles and folds in the cuff structure 6, allowing prevention of bias in a pressure distribution in the cuff structure 6.

In addition, in the blood pressure measurement device 1, the resin material that can be suitably thermally welded is used for the curler 5, the palm-side cuff 71, and the back-side cuff 74, allowing the curler 5, the palm-side cuff 71, and the back-side cuff 74 to be directly welded together. For this reason, for the curler 5 and the cuff structure 6, any of abutment portions between the curler 5 and the palm-side cuff 71 and the back-side cuff 74 may be welded again, eliminating a need for providing a bonding layer or providing a junction margin for bonding or sewing as illustrated in FIG. 11.

Additionally, as in known configurations, when the curler and cuff structure are joined using a bonding layer, the dimension in the thickness direction increases by the amount of the bonding layer. Furthermore, in a case where the curler and the cuff structure are joined using a junction method such as sewing or riveting, another member is required. Thus, the dimension in the width direction or the thickness direction are increased as that of the provided another member.

However, the curler 5 and the cuff structure 6 of the present embodiment can be joined by welding, preventing an increase in widthwise dimension or thickness-wise dimension resulting from junction with a bonding layer or another member. Thus, an increase in dimension caused by junction can be prevented. As a result, the blood pressure measurement device 1 can prevent an increase in the external shape of the curler 5 and cuff structure 6.

As a result, the blood pressure measurement device 1 can be miniaturized, and highly accurate blood pressure measurement can be stably performed for a long period of time.

Furthermore, the curler 5 is configured to be formed with a material similar to the resin material of the palm-side cuff 71 and the back-side cuff 74, enabling constitution with a single type of resin material to facilitate manufacturing. Additionally, all portions of the curler 5 can be thermally welded to the cuff structure 6, and thus the welding portion can be designed at a high degree of freedom.

Furthermore, the blood pressure measurement device 1 is configured such that the cuff structure is thermally welded to the curler 5, and thus the widthwise dimension of the cuff structure 6 may be equal to or smaller than the widthwise dimension of the curler 5. Thus, the cuff structure 6 can be disposed at the same position as that of the curler 5 or on an inner side of the curler 5, allowing the blood pressure measurement device 1 to be miniaturized.

This effect will be described in detail. For example, in a case where the blood pressure measurement device is joined using a bonding layer or another member, a junction margin is required. Even when a junction margin is secured by reducing the width of the cuff in the width direction of the curler 5, the external dimensions of the blood pressure measurement device may be increased. However, reducing the width of the cuff reduces the measurement accuracy in blood pressure measurement. However, since in the blood pressure measurement device 1 of the present embodiment, the cuff structure 6 and the curler 5 are thermally welded, the width of each of the cuffs 71 and 74 of the cuff structure 6 can be adjusted to the width of the curler 5. Thus, the cuff structure 6 can be joined to the curler 5, and the width of each of the cuffs 71 and 74 can be ensured, allowing the blood pressure measurement device 1 to be miniaturized with the measurement accuracy in blood pressure measurement being maintained.

As described above, the blood pressure measurement device 1 according to the present embodiment can be miniaturized by thermally welding, to the curler 5, the palm-side cuff 71 and the back-side cuff 74 constituted by a material similar to the material of the curler 5.

### Second Embodiment

Now, a second embodiment of the blood pressure measurement device 1 will be described using FIGS. 18 to 22. Note that the blood pressure measurement device 1 according to the second embodiment is configured such that a curler 5A includes a composite material, and differs, in this regard, from the blood pressure measurement device 1 according to the first embodiment described above in which the curler 5 is constituted by a single material. Thus, components of the blood pressure measurement device 1 of the second embodiment that are similar to the corresponding components of the blood pressure measurement device 1 according to the first embodiment described above are denoted by the same reference signs in the description, and descriptions and illustrations of these components are omitted as appropriate.

The blood pressure measurement device 1 according to the second embodiment includes the device body 3, the belt 4 that fixes the device body 3 to the wrist, the curler 5A disposed between the belt 4 and the wrist, the cuff structure 6 including the palm-side cuff 71, the sensing cuff 73, and the back-side cuff 74, and the fluid circuit 7 that fluidly connects the device body 3 and the cuff structure 6.

The curler 5A is constituted by a plurality of materials, and the portion of the curer 5A thermally welded to the cuff structure 6 is constituted by a material similar to the material of the cuff structure 6.

Specifically, the curler 5A is configured in a band-like shape that curves along the circumferential direction of the wrist. The curler 5A is formed with a first end and a second end spaced apart from each other. A first end-side outer surface of the curler 5A is fixed to the back lid 35 of the device body 3. The curler 5A is disposed at a position where the first end and the second end protrude from the back lid 35. Furthermore, the first end and the second end of the curler 5A are located adjacent to each other at a predetermined distance from each other.

As a specific example, the curler 5A is fixed to the living body side end portion of the outer case 31 or the base 33 along with the back lid 35 using the screws 35a or the like. Additionally, the curler 5A is fixed to the back lid 35 such that the first end and the second end of the curler 5A are located on one lateral side of the wrist 200 when the blood pressure measurement device 1 is attached to the wrist 200.

As a specific example, the curler 5A has a shape that curves along a direction orthogonal to the circumferential direction of the wrist, in other words, along the circumferential direction of the wrist 200 in a side view from the longitudinal direction of the wrist. The curler 5A extends, for example, from the device body 3 through the hand back side of the wrist 200 and the one lateral side of the wrist 200 to the hand palm side of the wrist 200 and toward the other lateral side of the wrist 200. In other words, by curving along the circumferential direction of the wrist 200, the curler 5A is disposed across the most of the wrist 200 in the circumferential direction, and both ends of the curler 5A are spaced apart from each other at a predetermined distance.

The curler 5A has hardness appropriate to provide flexibility and shape retainability. Here, “flexibility” refers to deformation of the shape of the curler 5A in the radial direction at the time of application of an external force of the belt 4 to the curler 5A. For example, “flexibility” refers to deformation of the shape of the curler 5A in a side view in which the curler 5A approaches the wrist, is along the shape of the wrist, or follows to the shape of the wrist when the curler 5A is pressed by the belt 4. Furthermore, “shape retainability” refers to the ability of the curler 5A to maintain a pre-imparted shape when no external force is applied to the curler 5A. For example, “shape retainability” refers to, in the present embodiment, the ability of the curler 5A to maintain the shape in a shape curving along the circumferential direction of the wrist.

The cuff structure 6 is disposed on an inner circumferential surface of the curler 5A, and is held along the shape of the inner circumferential surface of the curler 5A. As a specific example, the cuff structure 6 is fixed to the curler 5A by disposing the palm-side cuff 71 and the back-side cuff 74 on the inner circumferential surface of the curler 5A, and thermally welding the palm-side cuff 71 and the back-side cuff 74 to an outer circumferential surface or the inner circumferential surface of the curler 5A.

The curler 5A is constituted by a thermoplastic resin material. For example, the curler 5A includes a first portion 5a that includes a region to which the palm-side cuff 71 and the back-side cuff 74 are welded, and a second portion 5b other than the first portion 5a. The first portion 5a and the second portion 5b of the curler 5A are integrally formed by resin molding such as injection molding, for example.

The first portion 5a is set at least in the region to which the palm-side cuff 71 and the back-side cuff 74 are welded. The first portion 5a is constituted by a material similar to the material of the palm-side cuff 71 and the back-side cuff 74. As long as the first portion 5a can provide the function of the curler 5A together with the second portion 5b, and the palm-side cuff 71 and the back-side cuff 74 can be welded to the first portion 5a, the range, shape, and the like of the first portion 5a can be appropriately set.

The second portion 5b constitutes a portion of the curler 5A other than the first portion 5a. The second portion 5b is provided for obtaining flexibility and shape retainability of the curler 5A. The range, shape, and the like of the second portion 5b can be appropriately set as long as the second portion 5b can provide the function of the curler 5A together with the first portion 5a. For example, the second portion 5b is constituted by a material that is harder than the material of the first portion 5a and has a lower elastic modulus than the material of the first portion 5a.

For example, polypropylene, polyethylene terephthalate, or polyethylene naphthalate can be used as a material constituting the second portion 5b. Furthermore, the second portion 5b may be formed on a metal material such as a metal plate.

In the present embodiment, as an example in which the cuff structure 6 is welded to the inner circumferential surface side, the curler 5A has a dual layer structure in which the first portion 5a is provided on the outer circumferential surface side and the second portion 5b is provided on the inner circumferential surface side, as illustrated in FIG. 18.

Like the blood pressure measurement device 1 according to the first embodiment described above, the blood pressure measurement device 1 including the curler 5A configured as described above can be miniaturized and can stably perform highly accurate blood pressure measurement for a long period of time. Furthermore, the curler 5A is constituted by the composite material such that the portion of the curler 5A that is welded to the cuff structure 6 includes a material similar to the material of the welded portion of the cuff structure 6 and such that the other portion of the curler 5A includes a material different from the material of the welded portion of the cuff structure 6.

Such a configuration enables the curler 5A to be suitably thermally welded to the cuff structure 6 and allows easy acquisition of the flexibility and shape retainability required for the curler 5A. Furthermore, the first portion 5a allows the curler 5A to be thermally welded to the cuff structure 6, and the second portion 5b allows the function required for curler 5A to be obtained. Thus, the material of the second portion 5b can be appropriately selected according to the function required for the curler 5A. In this way, the curler 5A has a high degree of freedom for material selection.

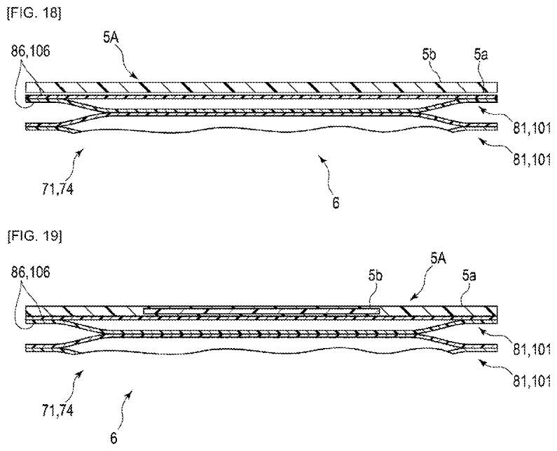

Note that the present invention is not limited to the embodiments described above. In the example described above, as an example in which the curler 5A is constituted by a composite material, the configuration having the dual layer structure has been described in which the first portion 5a is provided on the outer circumferential surface side and in which the second portion 5b is provided on the inner circumferential surface side. However, no such limitation is intended. For example, as another example, as illustrated in FIG. 19, the outer surface side of the curler 5A may be constituted by the first portion 5a, and the central side of the curler 5A may be constituted by the second portion 5b as a core material.

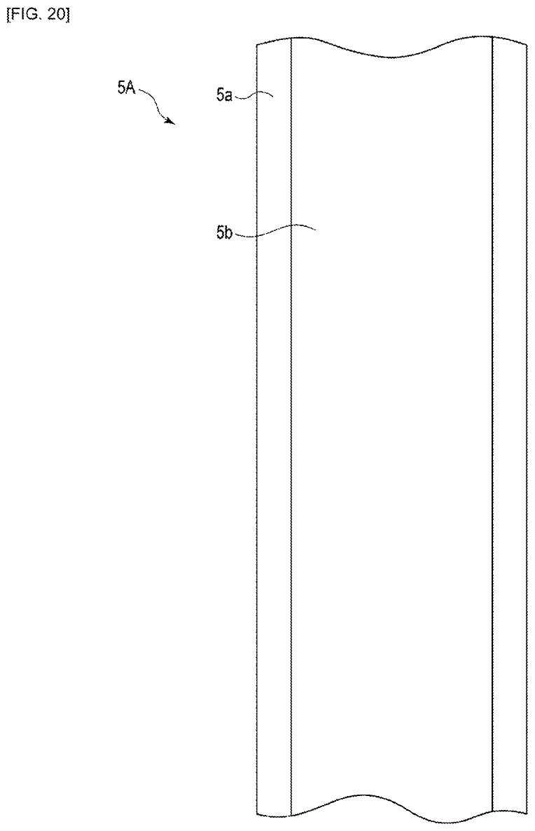

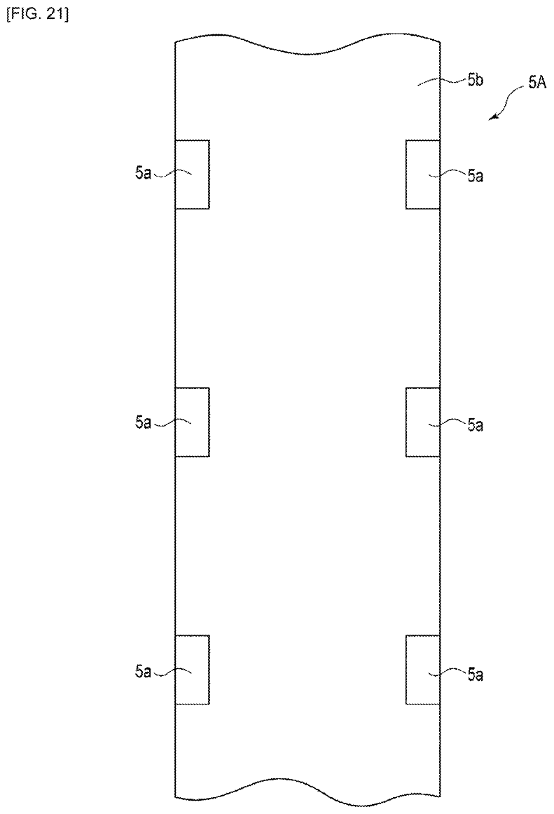

As another example, as illustrated in FIG. 20, the curler 5A and the cuff structure 6 may be configured to be welded at the edges of the curler 5A and the cuff structure 6 along the longitudinal direction, and both edges of the curler 5A along the longitudinal direction may be constituted by the first portion 5a, and the central side of the curler 5A in the longitudinal direction may be constituted by the second portion 5b. Furthermore, as illustrated in FIG. 21, the curler 5A may have a configuration in which a plurality of the first portions 5a are disposed in the portion where welding is performed.

As another example, the blood pressure measurement device 1 may be configured such that the palm-side cuff 71 and the back-side cuff 74 include junction pieces 99 that are disposed on the outer circumferential surface of the curler 5A and that are joined to the curler 5A, with the first portion 5a being disposed on the outer circumferential surface side of the curler 5A, as illustrated in FIG. 22.

The junction pieces 99 are constituted by, for example, setting the width of at least the first-layer sheet members 86 or 106 of the air bag 81 or 101 larger than the width of the curler 5A, and folding back two widthwise edges of the sheet members 86 or 106. In this manner, the junction piece 99 is constituted by a part of the cuff structure 6, and the junction piece 99 is folded back toward the outer circumferential surface of the curler 5A and joined. Thus, even in a case where the cuff structure 6 is welded on the outer circumferential surface of the curler 5A, an increase in the widthwise dimension of the curler 5A can be suppressed, enabling the blood pressure measurement device 1 to be miniaturized.

Additionally, the curler 5A and the air bags 81 or 101 may be configured to be welded on both sides of the curler 5 or 5A. As a specific example, in the blood pressure measurement device 1, both surfaces of the curler 5 or 5A are welded by providing the air bags 81 or 101 with the junction pieces 99, welding the junction pieces 99 and the outer circumferential surface of the curler 5A, and welding the sheet members 86 or 106 and the inner circumferential surface of the curler 5A. Such a configuration allows the curler 5 or 5A and the cuff structure 6 to be more firmly joined.

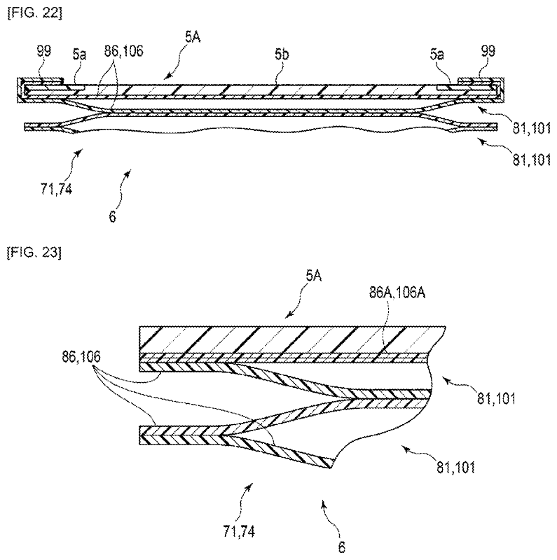

Additionally, in the example described above, the configuration has been described in which the curler 5A is provided with the first portion 5a constituted by a material similar to the resin material of the palm-side cuff 71 and the back-side cuff 74, but no such limitation is intended. For example, as another embodiment, of the sheet members 86 or 106 constituting the air bags 81 or 101 in the palm-side cuff 71 and the back-side cuff 74 welded to the curler 5, the sheet member 86 or 106 facing the curler 5 may be a sheet member 86A or 106A with a multilayer structure, as illustrated in FIG. 23, and the resin material on the curler 5 side of the sheet member 86A or 106A of the multilayer structure may be similar to the resin material of the curler 5.

For example, in the blood pressure measurement device 1, the timings when the first on-off valve 16A and the second on-off valve 16B are opened and closed during blood pressure measurement are not limited to the timings in the examples described above, and can be set as appropriate. Additionally, although the example has been described in which the blood pressure measurement device 1 performs blood pressure measurement by calculating the blood pressure with the pressure measured during the process of pressurizing the palm-side cuff 71, no such limitation is intended and the blood pressure may be calculated during the depressurization process or during both the pressurization process and the depressurization process.

In addition, in the example described above, the configuration has been described in which the air bag 81 is formed by each of the sheet members 86, but no such limitation is intended, and for example, the air bag 81 may further include any other configuration in order to manage deformation and inflation of the palm-side cuff 71, for example.

Additionally, in the examples described above, the configuration is described in which the back plate 72 includes the plurality of grooves 72a, but no such limitation is intended. For example, for management of the likelihood of deformation and the like, the number, the depth, and the like of the plurality of grooves 72a may be set as appropriate, and the back plate 72 may be configured to include a member that suppresses deformation.

Additionally, in the example described above, as the blood pressure measurement device 1, the configuration has been described in which the curler 5 or 5A and the cuff structure 6 are joined by thermal welding, but no such limitation is intended. For example, the blood pressure measurement device 1 may be configured such that in the manufacturing step of joining the cuff structure 6 to the curler 5 or 5A, a step may be executed in which the cuff structure 6 is temporarily fixed in advance to the curler 5 or 5A using a bonding layer of a double-sided tape or the like, the step being followed by welding. Additionally, the blood pressure measurement device 1 may be configured to join parts of the curler 5 or 5A and the cuff structure 6 using a bonding layer of a double-sided tape or the like, in addition to thermal welding. The blood pressure measurement device 1 configured as described above is configured to join the curler 5 or 5A and the cuff structure 6 by junction of temporary fixation using a bonding layer, partial junction, and thermal welding. Thus, this junction requires a smaller amount of bonding layer than junction using only the bonding layer. In addition, temporary fixation and partial junction can be performed at the abutment portion between the curler 5 or 5A and the cuff structure 6, thus eliminating the need to provide a separate junction margin for junction using a bonding layer. Thus, the blood pressure measurement device 1 can be miniaturized.

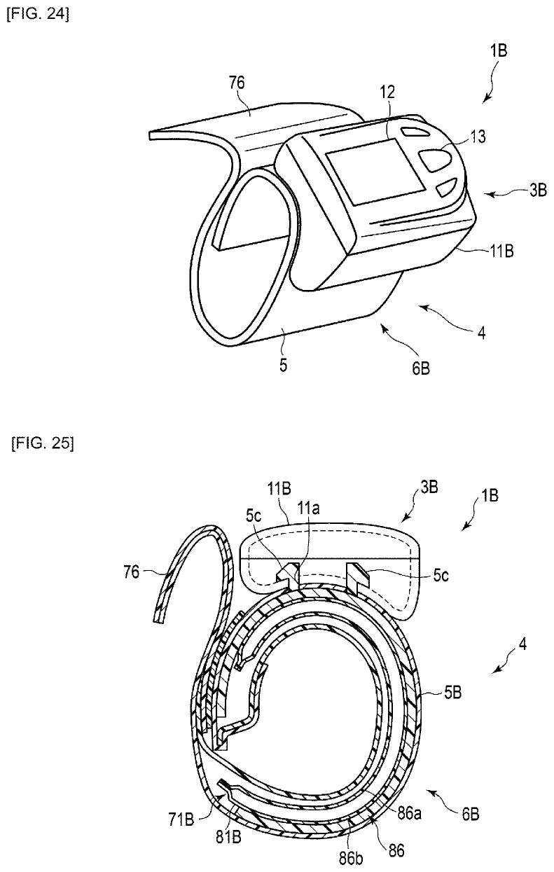

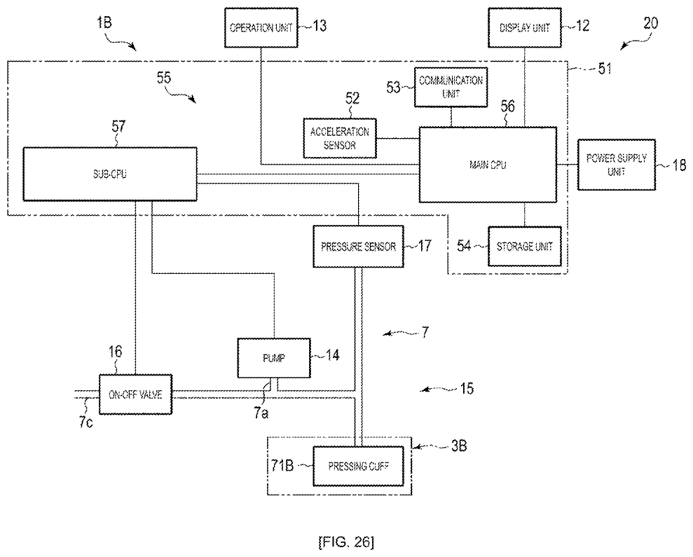

Furthermore, in the example described above, the blood pressure measurement device 1 has been described using an example of a wearable device attached to the wrist 200, but no such limitation is intended. For example, the blood pressure measurement device may be a blood pressure measurement device 1B wrapped around the upper arm to measure the blood pressure. Hereinafter, as a third embodiment, the blood pressure measurement device 1B will be described with reference to FIGS. 24 to 26. Note that components in the present embodiment that are similar to the corresponding components of the blood pressure measurement device 1 according to the first embodiment described above are denoted by the same reference signs in the description, and descriptions and illustrations of these components are omitted as appropriate.

For example, as illustrated in FIGS. 24 to 26, the blood pressure measurement device 1B in the third embodiment includes a device body 3B and a cuff structure 6B. The device body 3B includes, for example, a case 11B, the display unit 12, the operation unit 13, the pump 14, the flow path unit 15, the on-off valves 16, the pressure sensors 17, the power supply unit 18, and the control substrate 20. As illustrated in FIG. 26, the device body 3B includes one of each of the pump 14, the on-off valves 16, and the pressure sensors 17.

The case 11B is constituted, for example, in a box shape. The case 11B includes an attachment portion 11a that fixes the cuff structure 6B. The attachment portion 11a is an opening provided in a back surface of the case 11B, for example.

As illustrated in FIGS. 24 to 26, the cuff structure 6B includes a curler 5B constituted by a thermoplastic resin material, a pressing cuff 71B provided on the living body side of the curler 5B and constituted by a thermoplastic resin material, and a bag-like cover body 76 inside which the curler 5B and the pressing cuff 71B are disposed and which includes a cloth or the like. The cuff structure 6B is wrapped around the upper arm.

The curler 5B includes a protruding portion 5c fixed to the attachment portion 11a, for example.

The pressing cuff 71B includes an air bag 81B and a tube provided to the air bag 81B and fluidly connected to the flow path unit 15. The pressing cuff 71B is housed in the bag-like cover body 76 together with the curler 5B, and is joined to the inner surface of the curler 5B by thermal welding.

The air bag 81B is constituted in a rectangular shape that is long in one direction. The air bag 81B is constituted, for example, by combining two sheet members 86 that are long in one direction, and thermally welding edges of the sheet members 86. As a specific example, the air bag 81B includes a first sheet member 86a and a second sheet member 86b in this order from the living body side. The second sheet member 86b constitutes the air bag 81B along with the first sheet member 86a.

The air bags 81B in the curler 5B and the pressing cuff 71B are joined by welding. In addition, at least the welded portions of the curler 5B and the pressing cuff 71B are constituted by similar materials as is the case with the above-described curlers 5 and 5A and palm-side cuff 71 and back-side cuff 74.

In the blood pressure measurement device 1B configured as described above, the curler 5B and the pressing cuff 71B are joined by welding, and the resin materials of at least the welded portions of the curler 5B and the pressing cuff 71B include similar resin materials. In this configuration, like the blood pressure measurement device 1 according to the first embodiment described above, the blood pressure measurement device 1 can be miniaturized and perform highly accurate blood pressure measurement for a long period of time.

Additionally, in the example described above, the configuration has been described in which the back plate 72 is applied to the outer surface of the first sheet member 86a of the palm-side cuff 71 and to the living body side main surface of the sensing cuff 73 using an adhesive layer, a double-sided tape, or the like, but no such limitation is intended. In other words, the back plate 72 may be configured to be thermally welded to the palm-side cuff 71 and the sensing cuff 73, as is the case with the curler 5 according to the first embodiment or the curler 5A according to the second embodiment described above. For example, in such a configuration, all of the backplate 72 may be formed by a material similar to the material of the palm-side cuff 71 and the sensing cuff 73, as is the case with the curler 5, and at least the portion welded to the palm-side cuff 71 and the sensing cuff 73 may be formed of a material similar to the material of the palm-side cuff 71 and the sensing cuff 73, as is the case with the curler 5A. In addition, the back plate 72 may be configured to be thermally welded to one of the palm-side cuffs 71 or the sensing cuff 73, and joined to the other by being applied.

In other words, the embodiments described above are merely examples of the present invention in all respects. Of course, various modifications and variations can be made without departing from the scope of the present invention. Thus, specific configurations in accordance with an embodiment may be adopted as appropriate at the time of carrying out the present invention.

Note that the present invention is not limited to the embodiment, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE SIGNS LIST

1, 1B Blood Pressure measurement device
3, 3B Device body
4 Belt
5, 5A, 5B Curler
5a First portion
5b Second portion
5c Protruding portion
6, 6B Cuff structure
7 Fluid circuit
7a First flow path
7b Second flow path
7c Third flow path
11, 11B Case
11a Attachment portion
12 Display unit
13 Operation unit
14 Pump
15 Flow path unit
16 On-off valve
16A First on-off valve
16B Second on-off valve
17 Pressure sensor
17A First pressure sensor
17B Second pressure sensor
18 Power supply unit
19 Vibration motor
20 Control substrate
31 Outer case
31a Lug
31b Spring rod
32 Windshield
33 Base
35 Back lid
35a Screw
41 Button
42 Sensor
43 Touch panel
51 Substrate
52 Acceleration sensor
53 Communication unit
54 Storage unit
55 Control unit
56 Main CPU
57 Sub-CPU
61 First belt
61a First hole portion
61b Second hole portion
61c Buckle
61d Frame body
61e Prong
62 Second belt
62a Small hole
62b Third hole portion
71 Palm-side cuff (cuff)
71B Pressing cuff
72 Back plate
72a Groove
73 Sensing cuff
74 Back-side cuff (cuff)
76 Bag-like cover body
81, 81B Air bag (bag-like structure)
84 Guide unit
86, 86A Sheet member
86a First sheet member
86b Second sheet member
86b1 Opening
86c Third sheet member
86c1 Opening
86d Fourth sheet member
91 Air bag (bag-like structure)
92 Tube
93 Connection unit
96 Sheet member
96a Fifth sheet member
96b Sixth sheet member
99 Junction piece
101 Air bag (bag-like structure)
102 Tube
103 Connection portion
106, 106A Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106 g Thirteenth sheet member
106g1 Opening
106 h Fourteenth sheet member
106h1 Opening
106i Fifteenth sheet member
106i1 Opening
106j Sixteenth sheet member
106j1 Opening
106k Seventeenth sheet member
106k1 Opening
1061 Eighteenth sheet member
200 Wrist
210 Artery

1. A blood pressure measurement device comprising:
a cuff structure formed of a resin material and configured to be inflated with a fluid; and
a curler curved in such a manner as to follow along a circumferential direction of a portion of a living body where the blood pressure measurement device is attached, the curler being formed with a first end and a second end spaced apart from each other, the cuff structure being welded to the curler, and a portion of the curler where the cuff structure is welded being formed of a material similar to a resin material forming the cuff structure.

2. The blood pressure measurement device according to claim 1, wherein the curler is formed of a material similar to the resin material constituting the cuff structure.

3. The blood pressure measurement device according to claim 1, wherein the curler includes a first portion provided in a portion welded to the cuff structure, the first portion constituted by a material similar to the material constituting the cuff structure, and a second portion formed integrally with the first portion and constituted by a material harder than the material of the first portion.

4. The blood pressure measurement device according to claim 1, wherein the cuff structure is welded to an inner circumferential surface of the curler.

5. The blood pressure measurement device according to claim 1, wherein the cuff structure includes, at a widthwise edge, a junction piece welded to a part of an outer circumferential surface of the curler, and is disposed on an inner circumferential surface of the curler.

6. The blood pressure measurement device according to claim 1, further comprising a back plate to which the cuff structure is welded, the cuff structure extending in the circumferential direction of the portion of the living body where the blood pressure measurement device is attached, and at least a portion of the back plate where the cuff structure is welded being formed of a material similar to the resin material forming the cuff structure.
